(19) United States
(12) Patent Application Publication
(10) Pub. No.: US 2016/0038552 A1
(43) Pub. Date: Feb. 11, 2016

(54) IMPROVED COGNITIVE SUPPLEMENTS

(71) Applicant: BUCK INSTITUTE FOR RESEARCH ON AGING, Novato, CA (US)

(72) Inventors: Dale E. BREDESEN, Novato, CA (US); Varghese JOHN, San Francisco, CA (US)

(73) Assignee: Buck Institute for Research on Aging, Novato, CA (US)

(21) Appl. No.: 14/776,626

(22) PCT Filed: Mar. 13, 2014
(86) PCT No.: PCT/US2014/025573

§ 371 Date: Sep. 14, 2015

Related U.S. Application Data
(60) Provisional application No. 61/790,001, filed on Mar. 15, 2013.

(51) Int. Cl. A61K 36/258; A23L 1/303; A23L 1/30; A61K 36/41; A61K 36/68; A61K 36/16; A61K 9/00; A61K 31/51; A61K 31/197; A61K 31/519; A61K 31/714; A61K 31/522; A61K 31/4375; A61K 31/4748; A61K 31/55; A61K 31/685; A61K 31/14; A61K 31/194; A61K 31/7068; A61K 31/198; A61K 31/205; A61K 31/4015; A61K 31/202; A61K 31/593; A23L 1/302

(52) U.S. Cl. CPC A61K 36/258; A23L 1/302; A23L 1/303; A23L 1/3002; A61K 36/41; A61K 36/68; A61K 36/16; A61K 9/0053; A61K 9/0014; A61K 31/51; A61K 31/197; A61K 31/519; A61K 31/714; A61K 31/522; A61K 31/4375; A61K 31/4748; A61K 31/55; A61K 31/685; A61K 31/14; A61K 31/194; A61K 31/7068; A61K 31/198; A61K 31/205; A61K 31/4015; A61K 31/202; A61K 31/593; A23V 2002/00

(57) ABSTRACT

The invention generally provides supplements and methods of using the same to improve cognitive functions such as learning, memory, concentration, focus, attention, and mood.

### CROSS REFERENCE TO RELATED APPLICATIONS

This application claims the benefit under 35 U.S.C. §119(e) of U.S. Provisional Application No. 61/790,001, filed Mar. 15, 2013, which is incorporated by reference in its entirety.

### BACKGROUND

1. Technical Field

The invention relates generally to cognitive supplements and methods of use. More particularly, the invention relates to supplements for improving cognitive ability and mood in an individual.

2. Description of the Related Art

The brain is a complex organ balancing numerous chemical pathways in order to preserve neuronal and synaptic function and overall brain health. Considerable research has been performed worldwide on the effects of aging and, in particular, neurological and neuropsychiatric diseases, on brain health and function. There are numerous approaches known in the art to enhance mood and cognitive performance in normal individuals, including pharmaceutical interventions, aerobic exercise, and certain cognitive training programs. While much research has been focused on individual mechanisms in brain health using single agent pharmaceuticals or supplements, only a negligible fraction of the research efforts have addressed more than a single target at one time.

Moreover, research for improving cognitive abilities of otherwise cognitively normal, young and healthy subjects is noticeably absent. The few isolated compounds claiming one or more cognitive effects that have been subjected to well controlled (e.g., randomized, double blind, placebo controlled) clinical trials in relatively significant sample sizes (e.g., >50) have only shown clinical effect in selected populations (e.g., an older population, cognitively impaired, abnormal, or low normal sub-population), and may therefore have no significant effect in a healthy population of relatively wide age range. Thus, a large segment of the population is without a comprehensive cognitive supplement.

### BRIEF SUMMARY

The invention relates generally to supplements for improving cognitive ability and mood in an individual, kits and methods of using the same.

In various embodiments, supplements are provided that comprise: one or more vitamins selected from the group consisting of vitamin B and vitamin D; one or more alkaloids selected from the group consisting of caffeine, vinpocetine, and huperzine; and one or more herbs selected from the group consisting of Rhodiola rosea, Bacopa monnieri, Panax ginseng, and Gingko biloba.

In one embodiment, the vitamin D is vitamin D3.

In another embodiment, the supplement comprises one or more B vitamins selected from the group consisting of vitamin B1 (thiamine), vitamin B5 (panthothenic acid), vitamin B9 (folate), methylcobalamin, hydroxocobalamin, and cyanocobalamin.

In a particular embodiment, the supplement comprises the B vitamins:
thiamine, panthothenic acid, and methylcobalamin.

In a certain embodiment, the supplement comprises the B vitamins: thiamine, panthothenic acid, and hydroxocobalamin.

In a further embodiment, the supplement comprises the B vitamins: thiamine, panthothenic acid, and cyanocobalamin.

In an additional embodiment, the supplement comprises folate.

In a certain particular embodiment, the supplement comprises caffeine, vinpocetine, and huperzine.

In a certain embodiment, the supplement comprises caffeine, cyclopropylmethyl apovincaminate, and huperzine.

In an additional embodiment, the supplement comprises caffeine, cyclopropylmethyl apovincaminate, and galantamine.

In a particular embodiment, the supplement comprises caffeine, vinpocetine, and galantamine.

In one embodiment, the supplement comprises Rhodiola rosea, Bacopa monnieri, Panax ginseng, and Gingko biloba.

In a certain embodiment, the supplement further comprises one or more Omega-3 fatty acids.

In a further embodiment, the one or more Omega-3 fatty acids are selected from the group consisting of docosahexaenoic acid (DHA) and eicosapentaenoic acid (EPA).

In an additional embodiment, the supplement comprises DHA and EPA.

In one embodiment, the supplement comprises DHA.

In a certain embodiment, the supplement comprises EPA.

In a particular embodiment, the supplement further comprises a lipid or phospholipid.

In another embodiment, the lipid or phospholipid is L-alpha glycerylphosphorylcholine (Alpha-GPC), choline bitartarate, or citicholine.

In yet another embodiment, the lipid or phospholipid is Alpha-GPC.

In a further embodiment, the lipid or phospholipid is choline bitartarate.

In a certain embodiment, the lipid or phospholipid is citicholine.

In one embodiment, the supplement further comprises one or more amino acids.

In an additional embodiment, the supplement comprises the one or more amino acids selected from the group consisting of L-theanine, L-metheanine, L-carnitine, and acetyl L-carnitine (ALCAR).

In a particular embodiment, the supplement comprises the one or more amino acids selected from the group consisting of L-theanine and L-metheanine

In a certain embodiment, the supplement comprises L-theanine.

In an additional embodiment, the supplement comprises the one or more amino acids selected from the group consisting of L-carnitine and ALCAR.

In a particular embodiment, the supplement comprises ALCAR.

In a certain particular embodiment, the supplement further comprises aniracetam, piracetam, and pramiracetam.

In one embodiment, the supplement further comprises aniracetam, piracetam, or pramiracetam.

In a further embodiment, the supplement comprises aniracetam.

In an additional embodiment, the supplement further comprises magnesium threonate, magnesium glycinate, magnesium oxide, magnesium gluconate, or magnesium citrate.

In a certain embodiment, the supplement comprises magnesium threonate.

In various embodiments, supplements are provided that comprise thiamine, panthothenic acid, folate, methylcobalamin, ALCAR, vitamin D3, caffeine, vinpocetine, huperzine, Rhodiola rosea, Bacopa monnieri, Panax ginseng, Gingko biloba, DHA, Alpha-GPC, L-theanine, aniracetam, and magnesium threonate.

In another embodiment, the supplement is formulated as a single unit dosage form or as a combination of supplement components in a plurality of unit dosage forms.

In one embodiment, the dosage form selected from the group consisting of: solid, semi-solid, powder, liquid, effervescent, rapidly dissolving in liquid, sublingual, time release, chewable, gummy, gum, lozenges, encapsulated, and tablet.

In various embodiments, a method is provided for improving cholinergic neurotransmission in a subject comprising administering the subject the supplement of any one of the foregoing embodiments.

In various embodiments, a method is provided for improving monoaminergic neurotransmission in a subject comprising administering the subject the supplement of any one of the foregoing embodiments.

In various embodiments, a method is provided for improving synaptic formation or maintenance in a subject comprising administering the subject the supplement of any one of the foregoing embodiments.

In various embodiments, a method is provided for increasing the concentration or mental focus in a subject comprising administering the subject the supplement of any one of the foregoing embodiments.

In a particular embodiment, the subject has at least one symptom associated with attention deficit disorder (ADD) and attention deficit hyperactive disorder (ADHD), sensory integration disorder, any learning or attention disorder (e.g., dyslexia), any cognitive disorder, or other disorders associated with learning, memory, or cognitive performance.

In an additional embodiment, administration of the supplement to the subject results in a decrease in inattentiveness, over-activity, impulsivity, or a combination thereof.

In a further embodiment, the supplement is administered at least one, at least two, at least three, at least four, or at least five times a day.

In one embodiment, the supplement is self-administered.

In a certain embodiment, the supplement is orally administered.

In a particular embodiment, the supplement is formulated for transdermal administration.

In a particular related embodiment, the one or more supplement components are administered the same time or different times.

In an additional embodiment, the supplement is administered for at least one week, at least two weeks, at least one month, at least two months, at least three months, at least four months, at least five months, at least six months, at least one year or more.

In various embodiments, a kit is provided the comprises the supplement of any one of the foregoing embodiments.

In one embodiment, the supplement is packaged as a single formulation.

In a certain embodiment, the supplement is packaged as multiple-component formulations, wherein each supplement component is individually packaged.

In an additional embodiment, the supplement is formulated in a solid dosage form.

In a particular embodiment, the supplement is formulated in a liquid dosage form.

In one embodiment, the supplement is a multiple-component formulation comprising both solid and liquid dosage forms.

In various embodiments, a supplement is provided according to any one of the foregoing embodiments that has one or more of the purported roles or functions disclosed in Table 1.

### BRIEF DESCRIPTION OF THE SEVERAL VIEWS OF THE DRAWINGS

FIG. 1 shows beta-amyloid precursor protein (APP) is a transmembrane receptor that is a critical mediator of plasticity, and functions as a molecular switch: processing at the beta, gamma, and caspase sites produces 4 peptides that mediate synaptic inhibition, neurite retraction, caspase activation, and ultimately programmed cell death. These enhance forgetting, and inhibit memory formation and maintenance. Conversely, processing at the alpha site yields 2 peptides that mediate neurite extension, synaptic maintenance, inhibit programmed cell death, and support memory formation and maintenance. This switch features positive (anti-homeostatic; prionic loop) feedback. The current invention involves the identification of a combination of ingredients that supports the positive, i.e., memory formation, side of this molecular switch.

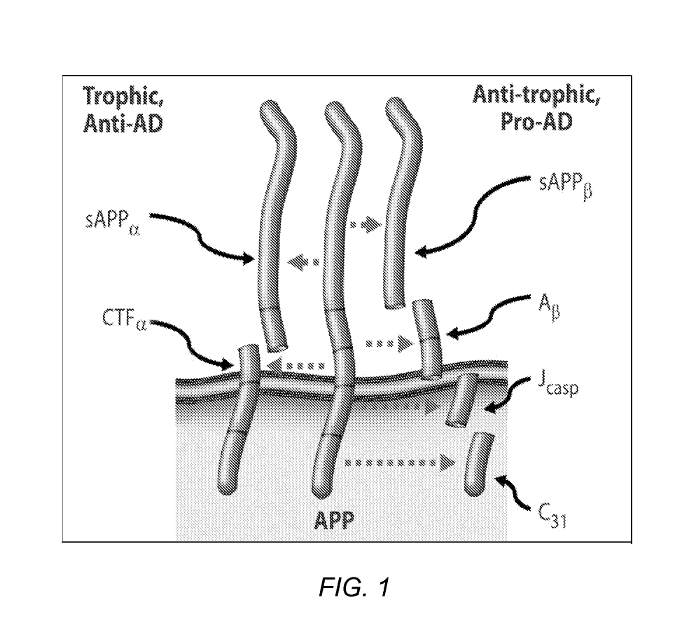

### DETAILED DESCRIPTION

### A. Overview

The present invention generally relates to supplements and compositions and methods of using the same to provide support for mental performance and/or improve cognitive abilities and/or mood. Existing supplements are mainly directed to help the elderly, those with neurological trauma, mild cognitive impairment (MCI) and/or neurodegenerative disease to regain some of the lost cognitive ability due to age or injury. Thus, existing compositions and methods fall far short of meeting the cognitive needs of the majority of the population.

The presently contemplated methods are directed, in part, to the use of the supplements contemplated herein to improve cognitive ability in cognitively normal, young, and otherwise healthy individuals, e.g., professionals, business executives, scientists, students, or those that want to improve cognitive function. In related embodiments, individuals may be young and otherwise healthy but also possess reduced cognitive ability due to various non-degenerative neurological disorders such as attention deficit disorder (ADD) and attention deficit hyperactive disorder (ADHD).

Supplements and compositions contemplated herein synergistically enhance an individual's overall cognitive ability by improving or enhancing short term working memory, long-term memory, mental attention, mental alertness, mental concentration or focus, learning, memory consolidation and processing speed, reaction time, mental clarity, mental energy, and general reasoning. Without wishing to be bound to any particular theory, it is further contemplated that the supplements disclosed herein increase cognitive ability and/or are associated with an improvement in moods such as depression, anxiety, confusion, hostility, and anger, thereby further expanding the capacity for an individual to improve their cognitive ability.

The present inventors have discovered a synergistic combination of vitamins, alkaloids, herbs, minerals, fatty acids, lipids and phospholipids, amino acids, and other compounds (e.g., racetams) that provide specific support factors for improving cognitive function and mood by increasing cholinergic and/or monoaminergic neurotransmission, promoting synapse formation, plasticity, and maintenance, and providing neuroprotective effects.

The practice of the invention will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); and Harlow and Lane, Antibodies, (Cold Spring Harbor Laboratory Press, Cold Spring Harbor, N. Y., 1998).

All publications, patents and patent applications cited herein are hereby incorporated by reference in their entirety.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein. For the purposes of the present invention, the following terms are defined below.

The articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

As used herein, the term “substantially” refers to a quantity, level, concentration, value, number, frequency, percentage, dimension, size, amount, weight or length that is 95%, 96%, 97%, 98%, 99% or 100% of a reference value. For example, a composition that is substantially free of a substance, e.g., a detergent, is 95%, 96%, 97%, 98%, 99% or 100% free of the specified substance, or the substance is undetectable as measured by conventional means. Similar meaning can be applied to the term “absence of,” where referring to the absence of a particular substance or component of a composition.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “supplement” refers to one or more compositions comprising the vitamins, alkaloids, herbs, minerals, fatty acids, lipids and phospholipids, amino acids, and other compounds as contemplated herein that individually or collectively improve cognitive ability and/or mood.

A “complete supplement” is one that contains all of the supplement components in one or more formulations. A complete supplement may be supplied in a single dosage form or as combinations of supplement components in one or more dosage forms.

As used herein, the term “supplement components” refers to the individual supplement ingredients, e.g., vitamins, alkaloids, herbs, minerals, fatty acids, lipids and phospholipids, amino acids, and other compounds (e.g., racetams) or compositions thereof.

As used herein, the phrase “a subject in need thereof” refers to a subject, as described infra, that would benefit from an improvement in cognitive ability and/or mood.

The terms “subject,” “individual,” and “patient” may be used interchangeably and refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments, the subject may not be under the care or prescription of a physician or other health worker. In various embodiments, the subject is about 10 years old to about 45 years old and otherwise cognitively normal and healthy. In one embodiment, the subject has or is at risk of having attention deficit disorder or attention deficit hyperactivity disorder.

An “effective amount” refers to an amount effective of a supplement or composition or component thereof, at dosages and for periods of time necessary, to achieve the desired result, e.g., an improvement in cognitive ability or mood.

A “therapeutically effective amount” of a supplement contemplated herein, may vary according to factors such as the disease state, age, sex, and weight of the individual, and the ability of the supplement to elicit a desired response in the individual. A therapeutically effective amount is also one in which any toxic or detrimental effects of a supplement are outweighed by the therapeutically beneficial effects. The term “therapeutically effective amount” refers to an amount of a supplement or composition that is effective to improve at least one aspect of cognitive ability in a mammal (e.g., an individual). In one embodiment, a therapeutically effective amount is an amount sufficient to improve short term working memory, long-term memory, mental attention, mental alertness, mental concentration or focus, learning, memory consolidation and processing speed, reaction time, mental clarity, mental energy, or general reasoning in an individual.

A “prophylactically effective amount” refers to an amount effective of a supplement or composition or component thereof, at dosages and for periods of time necessary, to achieve the desired result. Typically but not necessarily, a prophylactic dose is used in subjects prior to any cognitive decline.

“Treatment,” “treating,” or “treat” as used herein, includes improving any desirable effect on the cognitive abilities that can be effected by a supplement as contemplated herein, and may include even minimal changes or improvements in one or more cognitive abilities of an individual. Treatments also refer to delaying the onset of, retarding or reversing the progress of, reducing the severity of, or alleviating or preventing cognitive decline. “Treatment,” “treating,” or “treat” does not necessarily indicate complete eradication or cure of a non-degenerative neurological condition, or associated symptoms thereof. In one embodiment, treatment comprises improvement of at least one symptom of a non-degenerative neurological condition being treated. The improvement may be partial or complete. The subject receiving this treatment is any subject in need thereof. Improvement in cognitive ability may be measured using any method accepted in the art.

The term “mitigating” refers to reduction or elimination of one or more symptoms, or risk factors associated with cognitive decline, and/or the prevention of that pathology or disease.

As used herein, the terms “improving,” “promoting,” “enhancing,” “stimulating,” or “increasing” generally refer to the ability of a supplement contemplated herein to produce or cause a greater physiological response (i.e., measurable downstream effect), as compared to the response caused by either vehicle or a control molecule/composition or a previous response of the individual receiving the supplement. Such measurable physiological response include, without limitation, an improvement in cognitive ability or mood, e.g., short term working memory, long-term memory, mental attention, mental alertness, mental concentration or focus, learning, memory consolidation and processing speed, reaction time, mental clarity, mental energy, or general reasoning. The measurable physiological response is compared to normal, untreated, or control-treated individuals or a previous response of the individual receiving the supplement. For example, the physiological response may be increased by at least 5%, 10%, 20%, 30%, 40%, 50%, 60%, 70%, 80%, 90%, 100%, 125%, 150%, 175%, 200%, or greater. An “improved,” “increased,” “promoted” or “enhanced” response is typically a “statistically significant” response, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle (the absence of an agent) or a control composition or the response of the individual measured at an earlier time.

As used herein, the terms “retaining” or “maintaining,” or “retain” or “maintain”, generally refer to the ability of a supplement contemplated herein to produce or cause a physiological response (i.e., measurable downstream effect) that prevents the loss of cognitive ability. For example, supplements contemplated herein allow the subject to retain at least at least 75%, at least 80%, at least 85%, at least 90%, at least 95% or about 100% of the cognitive ability present in the subject prior to the subject being administered a supplement contemplated herein.

As used herein, the terms “decrease” or “lower,” or “lessen,” or “reduce,” or “abate” refers generally to the ability of a supplement contemplated herein to produce or cause a lesser physiological response (i.e., downstream effects), as compared to the response caused by either vehicle or a control molecule/composition, e.g., decreased neuronal cell death, or a previous response of the individual receiving the supplement. In one embodiment, the decrease can be a decrease in gene expression or a decrease in cell signaling that normally is associated with a reduction of cell viability. A “decrease” or “reduced” response is typically a “statistically significant” response, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle (the absence of an agent) or a control composition or a previous response of the individual receiving the supplement.

Cognition refers to how a person understands and acts in the world. It is a set of abilities, skills or processes that are part of nearly every human action. Cognitive abilities are the brain-based skills we need to carry out any task from the simplest to the most complex. They have more to do with the mechanisms of how we learn, remember, problem solve, and pay attention rather than with any actual knowledge. Cognitive abilities include, but are not limited to short term working memory, long-term memory, mental attention, mental alertness, mental concentration or focus, learning, memory consolidation and processing speed, reaction time, mental clarity, mental energy, or general reasoning.

Monoamine neurotransmitters are neurotransmitters and neuromodulators that contain one amino group that is connected to an aromatic ring by a two-carbon chain (—CH2-CH2-). All monoamines are derived from aromatic amino acids like phenylalanine, tyrosine, tryptophan, and the thyroid hormones by the action of aromatic amino acid decarboxylase enzymes. Illustrative examples of monoamine neurotransmitters include, but are not limited to histamine (His/H is diamine); catecholamines, e.g., dopamine, noradrenaline (norepinephrine), adrenaline (epinephrine); tryptamines e.g., serotonin (5-HT), melatonin; trace amines e.g., β-Phenylethylamine (PEA, β-PEA), tyramine, tryptamine, octopamine, 3-iodothyronamine; and thyronamines, a group of compounds derived from thyroid hormones.

In neuroscience and related fields, the term cholinergic is used in the following related contexts: a substance (or ligand) is cholinergic if it is capable of producing, altering, or releasing acetylcholine (“indirect-acting”) or mimicking its behavior at one or more of the body's acetylcholine receptor types (“direct-acting”); a receptor is cholinergic if it uses acetylcholine as its neurotransmitter; a synapse is cholinergic if it uses acetylcholine as its neurotransmitter. Two types of cholinergic receptors exist: nicotinic acetylcholine receptors (nAChR) and muscarinic acetylcholine receptor (mAChR). Both muscarinic and nicotinic receptors have been implicated in cognition and there is a convergence of evidence supporting the critical role of the cholinergic system in Alzheimer's disease: (a) Centrally active anticholinergic agents produce attention and memory deficits; (b) cholinergic neurotransmission modulates memory and learning; (c) lesions of the central cholinergic system create learning and memory impairments which are attenuated with cholinergic agents; and (d) postmortem studies of Alzheimer's patients consistently document cholinergic abnormalities with the degree of cognitive impairment.

### C. Supplements

In various embodiments, supplements disclosed herein improve cognitive ability, e.g., short term working memory, long-term memory, mental attention, mental alertness, mental concentration or focus, learning, memory consolidation and processing speed, reaction time, mental clarity, mental energy, and general reasoning, and/or moods by increasing structural and/or functional characteristics of the central nervous system, such as, for example, increasing cholinergic and/or monoaminergic neurotransmission, increasing synapse formation, increasing synaptic strength, increasing the maintenance of synapses, increasing neuronal cell survival, decreasing neuronal cell death, and/or providing neuroprotective effects.

Supplements and compositions contemplated herein include, but are not limited to, one or more vitamins, alkaloids, and herbs. Supplements may further comprise various minerals, fatty acids, lipids and phospholipids, amino acids and amino acid derivatives, and other compounds, such as, for example, racetams. Illustrative components of the supplements and compositions contemplated herein are provided infra.

1. Vitamins

Vitamin deficiencies are often associated with various forms of neurodegenerative disease or decreased cognition. As used herein the term “vitamin” includes a naturally occurring vitamin, a vitamin precursor, a salt derivative of a vitamin, a vitamin ester, or a metabolite thereof, either in a natural or synthetic form. Vitamins are inexpensive and generally well tolerated and have been found to improve a number of cognitive abilities. Examples of vitamins suitable for use in the supplements and compositions contemplated herein include, but are not limited to, one or more D or B vitamins.

In one embodiment, a supplement comprises one or more D vitamins and one or more B vitamins. In a particular embodiment, a supplement comprises one or more D vitamins or one or more B vitamins. Preferred examples of vitamin D include vitamin D3; preferred examples of vitamin B include vitamin B1, vitamin B5, vitamin B9, and vitamin B12.

a. Vitamin D

Vitamin D receptors are widespread in brain tissue. The biologically active form of vitamin D, vitamin D3 (cholecalciferol) is inexpensive and is a well tolerated dietary supplement that has anti-inflammatory and neuroprotective properties that improve learning, memory, and other cognitive abilities. Further, studies have shown associations between low vitamin D3 and individuals having neurodegenerative diseases, dementia, and cognitive impairment. In addition, two large prospective studies recently indicated that low vitamin D concentrations may increase the risk of cognitive decline. Thus, the potential therapeutic benefits of vitamin D3 may be considered at least two-fold, increasing cognitive abilities while at the same time reducing or preventing age-related cognitive decline.

Existing commercial sources of vitamin D3 may be used in particular embodiments, e.g., Jarrow, Nordic Naturals, NatureMade, Puritan, Pure Encapsulations, Beyond Health, and other standard commercial suppliers.

Preferred amounts of vitamin D3 used within supplements and compositions of the invention include about 500 IU to about 5000 IU, about 750 IU to about 5000 IU, or about 1000 IU to about 5000 IU, or any intervening range therein. In particular preferred embodiments, a supplement or composition comprises about 500 IU, about 750 IU, about 1000 IU, about 1500 IU, about 2000 IU, about 3000 IU, about 4000 IU, or about 5000 IU, or any intervening amount therein.

b. B-Vitamins

B-complex vitamins play both direct and indirect roles in maintaining optimal neurological function. B-complex vitamins have been found to act as acetylcholine synthesis co-factors. Accordingly, the presence of B-complex vitamins may increase acetylcholine synthesis and positively affect neural function.

B vitamins also play an indirect role in cognitive function by optimizing the levels of methylation and thereby reducing toxic levels of homocysteine (byproduct of normal amino acid metabolism). Homocysteine toxicity can result in decreased neural and systemic oxygenation, increased free radical pathology, arteriosclerosis, cancer, neuro-vascular decline, and neurodegenerative disorders. Pathological levels of homocysteine are also a marker for memory loss, cognitive dysfunction and Alzheimer's disease. In addition, studies show that people with the highest blood levels of B-complex vitamins score highest on tests of cognitive function.

Thus, specific B-complex vitamin supplements improve cognitive function, focus, concentration, alertness, and memory by promoting synaptic neurotransmission, optimal methylation and reducing toxic levels of homocysteine. Existing commercial sources of B-complex vitamins (B multivitamin) may be used in particular embodiments to achieve the desired amounts of individual B vitamins, e.g. Nature's Way, Nature Made, Beyond Health, Jarrow, Pure Encapsulations, GNC, etc.

i. Vitamin B1 (Thiamine)

Thiamine is required for the production of multiple enzymes in glucose metabolism in the brain. Thiamine can mimic the activities of acetylcholine—the major learning neurotransmitter associated with attention, concentration and memory and can block tau phosphorylation, which is a marker for neurodegenerative disease. Thiamine deficiency leads to memory loss, for example in Wenicke-Korsakov syndrome. Increased thiamine consumption is associated with improved cognitive function, reduced mental fatigue, and faster reaction times. Existing commercial sources of thiamine may also be used in particular embodiments, e.g. Jarrow, Nature Made, Puritan, Scout, and other standard suppliers of thiamine.

Preferred amounts of thiamine used within supplements and compositions of the invention include about 2.5 mg to about 25 mg, about 5 mg to about 25 mg, or about 10 mg to about 25 mg, or any intervening range therein. In particular preferred embodiments, a supplement or composition comprises about 2.5 mg, about 5.0 mg, about 10 mg, about 15 mg, about 20 mg, or about 25 mg, or any intervening amount therein.

ii. Vitamin B5 (Pantothenic Acid; Pantothenate)

Pantothenic acid is required for the synthesis of both acetyl CoA, which is involved in cellular metabolism, and acetylcholine, which is important for cholinergic synaptic transmission at the acetylcholine receptor. Pantothenic acid also supports alertness and attention. Thus, supplementation with pantothenic acid supports neuronal health, strengthens cholinergic synapses and increases cholinergic synaptic transmission thereby improving cognitive function, focus, mental alertness, concentration, and memory. Supplements and compositions of the present invention may comprise natural or synthetic pantothenic acid.

Pantothenic acid may be supplied in various forms, such as, for example, calcium pantothenate.

Preferred amounts of pantothenic acid used within supplements and compositions of the invention include about 100 mg to about 250 mg, about 100 mg to about 200 mg, or about 150 mg to about 250 mg, or any intervening range therein. In particular preferred embodiments, a supplement or composition comprises about 100 mg, about 150 mg, about 200 mg, or about 250 mg or any intervening amount therein.

iii. Vitamin B9 (Folic Acid; Folate)

Folic acid is a collective term for pteroylglutamic acids and their oligoglutamic acid conjugates. Folic acid is itself not biologically active, but its biological importance is due to tetrahydrofolate and other derivatives after its conversion to dihydrofolic acid in the liver. Supplemental folic acid is important for cellular metabolism in men, women and children of all ages. Folate is required for DNA synthesis and repair, as a co-factor in particular biological reactions, and for production of red blood cells.

Folate deficiencies have been found to be associated with irritability, depression, poor cognitive function and memory loss and increased levels of folate decrease homocysteine levels. Thus, folate supplementation may improve cognitive function, focus, concentration, mental alertness, and memory by reducing toxic levels of homocysteine.

Folate may be supplied in various forms, such as, for example, methyl-folate or 5-methyl-tetra-hydrofolate.

Preferred amounts of folate used within supplements and compositions of the invention include about 0.4 mg to about 10 mg, about 0.8 mg to about 2.5 mg, or about 1.5 mg to about 5 mg, or any intervening range therein. In particular preferred embodiments, a supplement or composition comprises about 0.4 mg, about 0.5 mg, about 0.6 mg, about 0.7 mg, about 0.8 mg, about 0.9 mg, about 1.0 mg, about 1.1 mg, about 1.25 mg, about 1.5 mg, about 1.75 mg, about 2.0 mg, about 2.25 mg, or about 2.5 mg, any intervening amount therein.

iv. Vitamin B12

Vitamin B-12 (cobalamin) refers to a group of cobalt-containing vitamins including but not limited to, cyanocobalamin, hydroxocobalamin, and methylcobalamin. Vitamin B12 is important for proper cognitive function because it helps maintain optimal levels of methylation, production of healthy blood, production of healthy myelin in neurons, and helps to decrease toxic homocysteine levels. Vitamin B12 deficiencies are common and even marginal deficiencies may result in depression, decreased brain volume, and cognitive decline. Thus, vitamin B12 supplementation may improve cognitive function, focus, concentration and memory by reducing toxic levels of homocysteine, preventing damage to neuronal cells, and promoting neuronal survival.

Vitamin B12 may be supplied in various forms, such as, for example, cyanocobalamin, hydroxocobalamin, and methylcobalamin. Because many people have defects in methylation of B12, methylcobalamin is preferred.

Preferred amounts of vitamin B12 used within supplements and compositions of the invention include about 0.5 mg to about 10 mg, about 2.5 mg to about 7.5 mg, or about 2.5 mg to about 10 mg, or any intervening range therein. In particular preferred embodiments, a supplement or composition comprises about 0.5 mg, about 0.6 mg, about 0.7 mg, about 0.8 mg, about 0.9 mg, about 1.0 mg, about 1.1 mg, about 1.2 mg, about 1.3 mg, about 1.5 mg, or about 1.5 mg, or any intervening amount therein.

2. Alkaloids

Alkaloids are a group of naturally occurring chemical compounds that contain mostly basic nitrogen atoms. This group also includes some related compounds with neutral and even weakly acidic properties. Some synthetic compounds of similar structure are also attributed to alkaloids. In addition to carbon, hydrogen and nitrogen, alkaloids may also contain oxygen, sulfur and more rarely other elements such as chlorine, bromine, and phosphorus.

Alkaloids are produced by a large variety of organisms, including bacteria, fungi, plants, and animals, and are part of the group of natural products (also called secondary metabolites). Many alkaloids can be purified from crude extracts by acid-base extraction. Many alkaloids are toxic to other organisms. Alkaloids act on a diversity of metabolic systems in humans and other animals and possess various pharmacological effects. Particular alkaloids have been shown to improve a number of cognitive abilities.

Illustrative examples of alkaloids suitable for use in the supplements and compositions contemplated herein include, but are not limited to caffeine, vinpocetine, cyclopropylmethyl apovincaminate, huperzine A, and galantamine. In one embodiment, a supplement comprises caffeine, vinpocetine, cyclopropylmethyl apovincaminate, huperzine A, and/or galantamine (huperzine A or galantamine). In a particular embodiment, a supplement comprises one or more of caffeine, vinpocetine, cyclopropylmethyl apovincaminate, huperzine A, or galantamine

a. Caffeine

Caffeine is a bitter, white crystalline xanthine alkaloid that acts as a stimulant drug. Caffeine is found in varying quantities in the seeds, leaves, and fruit of the coffee plant, tea bush, kola nut, verba maté, guarana berries, guayusa, and yaupon holly. The effects of caffeine on cognition include an increase in learning and memory tasks, increased mental alertness, reaction time, and reduced mental fatigue. Caffeine has also been reported to prevent cognitive decline in healthy subjects. Caffeine's ability to improve memory and cognition may stem from its ability to increase of neurotrophins and/or neurotrophin receptors that promote increase in cognitive function, e.g., increasing the amount of BDNF and TrkB in the hippocampus.

In particular embodiments, supplements and compositions of the invention comprise natural or synthetic caffeine, or Guarana extract. Existing commercial sources of caffeine may also be used in particular embodiments, e.g. ProLab, Purebulk, Amazon, GNC, or other standard sources of caffeine.

Preferred amounts of caffeine used within supplements and compositions of the invention include about 25 mg to about 200 mg, about 25 mg to about 100 mg, or about 50 mg to about 75 mg, or any intervening range therein. In particular preferred embodiments, a supplement or composition comprises about 25 mg, about 30 mg, about 35 mg, about 40 mg, about 45 mg, about 50 mg, about 55 mg, about 60 mg, about 65 mg, about 70 mg, or about 75 mg, or any intervening amount therein.

b. Vinpocetine

Vinpocetine is a semisynthetic derivative alkaloid of vincamine (ethyl apovincaminate), a Vinca minor (periwinkle) extract. Cyclopropylmethyl apovincaminate is a synthetic cyclic ester derivative of vincamine. Vinpocetine improves blood flow, circulation and oxygen utilization in the brain of animals and humans and boosts memory in young, healthy individuals. In addition, vinpocetine is considered a nontoxic herbal extract and has been well tolerated in various clinical studies. Vinpocetine's anti-inflammatory properties may increase stress-induced neuronal survival. Vinpocetine has been shown to selectively inhibit voltage-sensitive Na+ channels and thereby provide a general neuroprotective effect through blockade of excitotoxicity and attenuation of neuronal damage induced by cerebral ischemia/reperfusion. In addition, several clinical studies conducted in England showed that vinpocetine increases cognitive performance and memory in both health and diseased individuals. Hindmarch I, et al. International Clinical Psychopharmacology, 6 (1): 31-43, Spring 1991; Subhan Z, and Hindmarch I, European Journal of Clinical Pharmacology, 28 (5): 567-571, 1985; and Coleston D M, Hindmarch I, Drug Dev. Res., 14: 191-193, 1988.

In particular embodiments, supplements and compositions of the invention comprise natural or synthetic vinpocetine and/or cyclopropylmethyl apovincaminate, and/or Vinca minor extract, e.g., Jarrow, Puritan, Banyan, Sahelian, etc.

Preferred amounts of vinpocetine used within supplements and compositions of the invention include about 2 mg to about 10 mg, about 5 mg to about 10 mg, or about 2 mg to about 7.5 mg, or any intervening range therein. In particular preferred embodiments, a supplement or composition comprises about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, or about 7.5 mg, or any intervening amount therein.

Other nootropic agents may also be included, such as aniracetam, piracetam, and pramiracetam. As an example, aniracetam, is used at 500 mg to 2500 mg, total per day, taken in 2 or 3 equal doses. Aniracetam may be used from IAS, Vitabrain, or other standard suppliers.

c. Huperzine A

Huperzine A (“huperzine”) is an alkaloid derived from the club moss Huperzia serrata. Huperzine has historically been used in Chinese medicine to treat inflammation and fever. Recently, huperzine was found to improve cognitive function, mental alertness, focus, concentration, and memory. The beneficial effects of huperzine supplementation may be linked to its ability to enhance or improve cholinergic transmission and by naturally decreasing acetylcholine hydrolysis through acetylcholinesterase inhibition and by increasing neuronal cell survival and decreasing neuronal cell death.

Huperzine is a preferred component in particular supplements contemplated by the present invention, in part, because it has demonstrated good penetration through the blood brain barrier, high oral bioavailability, and long durations of acetylcholinesterase inhibition. In addition, huperzine appears to produce its cognitive improvements with fewer side effects and longer duration than current drugs which perform in much the same manner.

Huperzine suitable for supplements and compositions of the invention include both natural or synthetic huperzine and Huperzia serrata extracts. Existing commercial sources of huperzine may also be used in particular embodiments, e.g. Source Naturals, Pure Formula, GNC, or other standard suppliers.

Preferred amounts of huperzine used within supplements and compositions of the invention include about 10 μg to about 200 μg, about 25 μg to about 100 μg, or about 50 μg to about 75 μg, or any intervening range therein. In particular preferred embodiments, a supplement or composition comprises about 25 μg, about 30 μg, about 35 μg, about 40 μg, about 45 μg, about 50 μg, about 55 μg, about 60 μg, about 65 μg, about 70 μg, or about 75 μg, or any intervening amount therein.

d. Galantamine

Galantamine, also known as galanthamine or (4aS,6R,8aS)-4a,5,9,10,11,12-hexahydro-3-methoxy-11-methyl-6H-benzofuro[3a,3,2-ef][2]benzazepin-6-ol, is a naturally occurring alkaloid, which can be prepared synthetically or may be derived from It is an alkaloid that is obtained synthetically or from the bulbs and flowers of snow drop species, Galanthus caucasicus, Galanthus nivalis, and Galanthus woronowii and related genera like Narcissus, Leucojum, and Lycoris. Galantamine has been used to treat a variety of conditions: arthritis, fatigue syndromes, mania, schizophrenia, memory dysfunction, Alzheimer's Disease, alcoholism, nicotine dependence, disorders of attention, and jet lag.

Galantamine has acetylcholinesterase inhibitory activity and is a reversible and competitive cholinesterase inhibitor. Thus, galantamine supplementation may enhance cognitive function, focus, concentration, mental alertness, and memory through improving cholinergic synaptic transmission and function by increasing the time that acetylcholine is available at the synapse.

Galantamine has a similar mechanism of action to huperzine A, and therefore, if both are used, dosage for each should be halved. If only galantamine is included, dosages are given below. Galantamine suitable for supplements and compositions of the invention include both natural or synthetic galantamine and Galanthus caucasicus, Galanthus nivalis, or Galanthus woronowii extracts. Existing commercial sources of galantamine may also be used in particular embodiments.

Preferred amounts of galantamine used within supplements and compositions of the invention include about 2 mg to about 24 mg, about 5 mg to about 15 mg, or about 5 mg to about 25 mg, or any intervening range therein. In particular preferred embodiments, a supplement or composition comprises about 2 mg, about 4 mg, about 6 mg, about 8 mg, about 10 mg, about 12 mg, about 14 mg, about 18 mg, about 20 mg, about 22 mg, or about 24 mg, or any intervening amount therein.

3. Herbs

As used herein, the term “herb” refers to a fresh or dried part of a plant or a whole plant or an extract thereof, which comprises a biological activity. Various methods are known for the production of therapeutic extracts from herbs. For example, herbs may be subjected to a polar (e.g., aqueous) solvent extraction. The aqueous extract may then be filtered if necessary to remove large particles, and subsequently dried or lyophilized. It is possible to use dry herbs directly by grinding to a powder. A number of herbs, herbal tinctures and herbal extracts are available from commercial suppliers.

Illustrative examples of herbs suitable for use in the supplements and compositions contemplated herein include, but are not limited to Rhodiola rosea, Bacopa monnieri, Ginkgo biloba, and Panax ginseng. In one embodiment, a supplement comprises one or more of Rhodiola rosea, Bacopa monnieri, Ginkgo biloba, and Panax ginseng, or extracts thereof. In a certain embodiment, a supplement comprises Rhodiola rosea, Bacopa monnieri, Ginkgo biloba, and Panax ginseng, or extracts thereof.

a. Rhodiola rosea

Rhodiola rosea is commonly known as “golden root,” “Arctic root,” or “Crenulin.” Rhodiola rosea is endogenous to the high altitudes of the Artic and mountainous regions of Europe and Asia. It is traditionally used in Eastern Europe and Asia to stimulate the nervous system, enhance physical and mental performance, and treat fatigue, psychological stress and depression. Studies have shown that Rhodiola rosea extract improves learning and memory, reduces cognitive dysfunction, and protect against neuronal injury from oxidative stress in animal models. In addition, Rhodiola rosea extract given to young, healthy individuals improved mental alertness, associative thinking, short-term memory, calculation and ability of concentration, speed of audio-visual perception, and reduced mental fatigue. Other studies have shown that Rhodiola rosea extract may improve cognitive ability and reduce fatigue by inhibiting monoamine oxidases (MAOs A and B).

Refined Rhodiola rosea can be prepared by known methods. In various embodiments, Rhodiola rosea is in the form of an extract, e.g., a standardized extract including 0.5% to 3.0% rosavins. In particular embodiments, supplements and compositions of the invention comprise existing commercial sources of Rhodiola rosea extract, e.g. Banyan, Solaray, Gaia, IAS, Sahelian, or other standard sources of Rhodiola rosea extract.

Preferred amounts of Rhodiola rosea (extract standardized to about 0.5% to about 8.0% rosavins) used within supplements and compositions of the invention include about 50 mg to about 1000 mg, about 100 mg to about 500 mg, or about 250 mg to about 500 mg, or any intervening range therein. In particular preferred embodiments, a supplement or composition comprises about 50 mg, about 100 mg, about 200 mg, about 300 mg, about 400 mg, about 500 mg, about 600 mg, about 700 mg, about 800 mg, about 900 mg, or about 1000 mg, or any intervening amount therein.

b. Bacopa monnieri (Also Referred to as Bacopa monniera)

Bacopa monniera is a traditional Ayurvedic herb utilized in India for more than 3,000 years to treat ulcers, tumors, ascities, enlarged spleen, indigestion, inflammations, leprosy, anemia, and biliousness. Bacopa monniera is also used to enhance memory capacity, improve intellectual and cognitive functions, reduce stress-induced anxiety and increase concentration. Two active compounds have been isolated from Bacopa monniera extracts were shown to enhance both short-term and long-term memory and regulate and restore proper synaptic activity in over-stimulated neurons. Bacopa monniera extracts may facilitate the acquisition, consolidation, retention, and recall of learned tasks by increasing kinase function to promote new protein synthesis of the brain cells involved with learning and memory. Bacopa monniera also possesses antioxidant properties reduce or prevent neuronal damage due to oxidative stress.

Bacopa extracts from the leaves of Bacopa monniera can be prepared by known methods. In particular embodiments, existing commercial sources of Bacopa extracts may be used in supplements and compositions of the invention, e.g., Banyan, Sahelian, Natura, Thorne, etc.

Preferred amounts of Bacopa monniera used within supplements and compositions of the invention include about 50 mg to about 500 mg, about 100 mg to about 500 mg, or about 200 mg to about 500 mg, or any intervening range therein. In particular preferred embodiments, a supplement or composition comprises about 50 mg, about 100 mg, about 150 mg, about 200 mg, about 250 mg, about 300 mg, about 350 mg, about 400 mg, or about 500 mg, or any intervening amount therein.

c. Ginkgo biloba

Ginkgo biloba is a unique species of tree with no close living relatives. Ginkgo biloba has been used medicinally for thousands of years. One standardized preparation of the Ginkgo leaf extract (EGb 761) contains two main bioactive constituents, flavonoid glycosides (24%) and terpene lactones (6%), along with less than 5 ppm of the allergenic component, ginkgolic acid. The Ginkgo leaf extract has been reported to have neuroprotective, anticancer, cardioprotective, stress alleviating, and memory enhancing effects and possible effects on tinnitus, geriatric complaints, and psychiatric disorders. Without being bound to any particular theory, the Ginkgo leaf extract's therapeutic properties are thought to arise from its antioxidant, antiplatelet, antihypoxic, antiedemic, hemorrheologic, and microcirculatory actions, where the flavonoid and the terpenoid constituents may act in a complementary manner.

Ginkgo leaf extract enhances cognitive function in healthy individuals and has been shown to increase levels of the monoaminergic neurotransmitters dopamine and noradrenaline, and also the cholinergic neurotransmitter acetylcholine, in a dose-dependent manner. Ginkgo leaf extract may provide these effects, in part, by inhibiting neurotransmitter uptake. Thus, the direct involvement of Ginkgo leaf extract in the increase of dopaminergic and cholinergic neurotransmission may be responsible for improving cognitive function.

Ginkgo leaf extract can be prepared by known methods. In particular embodiments, existing commercial sources of Ginkgo leaf extracts may be used in supplements and compositions of the invention, e.g., Banyan, GNC, Vitaminshoppe, IAS, etc.

Preferred amounts of Ginkgo biloba used within supplements and compositions of the invention include about 10 mg to about 400 mg, about 25 mg to about 200 mg, or about 60 mg to about 120 mg, or any intervening range therein. In particular preferred embodiments, a supplement or composition comprises about 10 mg, about 20 mg, about 30 mg, about 40 mg, about 50 mg, about 60 mg, about 70 mg, about 80 mg, about 90 mg, about 100 mg, about 110 mg, or about 120 mg, or any intervening amount therein.

d. Panax ginseng

Panax ginseng is a shade-loving, deciduous perennial with five-fingered leaves, tiny white flowers, red berries, and a yellowish-brown root. The root is utilized medicinally, although active compounds are present in all other parts of the plant. Panax ginseng, used medicinally for thousands of years in China, Korea, and Japan, is well known as an adaptogen and a restorative tonic that is widely used in traditional Chinese medicine and Western herbal preparations. Eclectic uses for Panax ginseng include fatigue, infertility, liver disease, amnesia, colds, menopause, and erectile dysfunction.

Recent evidence suggests that standardized Panax ginseng extract can improve certain aspects of cognitive performance and mood in healthy young volunteers in a dose and time dependent manner. For example, ginseng improves speed of attention, indicating a beneficial effect on an individual's ability to allocate attentional processes to a particular task. Ginseng may further improve mental alertness, concentration, and memory.

Panax ginseng extract can be prepared by known methods. In particular embodiments, existing commercial sources of Panax ginseng extracts may be used in supplements and compositions of the invention, e.g., Banyan, Puritan's Pride, GNC, Vitaminshoppe, etc.

Preferred amounts of Panax ginseng used within supplements and compositions of the invention include about 100 mg to about 1000 mg, about 200 mg to about 800 mg, or about 300 mg to about 600 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, or about 1000 mg, or any intervening amount therein.

4. Minerals

Minerals are another category of underrated neuro-nutrients that play vital roles in mental function. Normal brain function is dependent on several key minerals that make up only 0.5 percent of the brain by weight. As used herein, the term “mineral” refers to an element or chemical compound that is typically a naturally occurring solid chemical substance formed through biogeochemical processes, having characteristic chemical composition, highly ordered atomic structure, and specific physical properties. Minerals as used herein include isolated minerals, or synthetically produced salts thereof. An illustrative example of minerals or elements suitable for use in the supplements and compositions contemplated herein includes, but is not limited to magnesium.

In one embodiment, a supplement comprises one or more of Mg threonate, Mg glycinate, Mg gluconate, Mg citrate, and Mg oxide.

a. Magnesium

Magnesium (Mg) is the fourth most abundant ion in body and a cofactor for more than 300 enzymes, is essential for the proper functioning of many tissues and organs, including the cardiovascular, neuromuscular, and nervous systems. In brain, one major action of Mg is modulating the voltage-dependent block of NMDA receptors (NMDAR), controlling their opening during coincidence detection that is critical for synaptic plasticity. Recently, magnesium compounds have been developed having high bioavailability, stability and blood brain barrier permeability, e.g., Mg threonate, Mg glycinate, Mg gluconate, Mg citrate, and Mg oxide. These Mg supplements were found to enhance synaptic plasticity, learning abilities, and short- and long-term memory in animal studies.

Magnesium may be supplied in various forms, such as, for example, Mg threonate, Mg glycinate, Mg gluconate, Mg citrate, and Mg oxide. Existing commercial sources of Magnesium may be used in supplements and compositions of the invention, e.g., Life Extension Foundation, Vitaminshoppe, Nature Made, etc.

Preferred amounts of magnesium used within supplements and compositions of the invention include about 50 mg to about 1000 mg, about 100 mg to about 800 mg, or about 250 mg to about 750 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, or about 1000 mg, or any intervening amount therein.

5. Fatty Acids

Omega-3 fatty acids are fats commonly found in marine and plant oils. They are polyunsaturated fatty acids with a double bond (C═C) starting after the third carbon atom from the end of the carbon chain. N-3 fatty acids may have health benefits and are considered essential fatty acids, meaning that they cannot be synthesized by the human body but are important for normal metabolism.

Illustrative examples of omega-3 fatty acids suitable for use in the supplements and compositions contemplated herein include, but are not limited to docosahexaenoic acid (DHA) and eicosapentaenoic acid (EPA). In one embodiment, a supplement comprises DHA and EPA; in a particular embodiment, a supplement comprises DHA or EPA.

DHA and EPA are orthomolecular, conditionally essential nutrients that are importance for neuronal synapse formation and maintenance. The most abundant omega-3 fatty acid present in the brain is DHA. DHA is concentrated in the synaptic gaps between axons and dendrites, where neural communication takes place. It is also abundant in the neuronal mitochondria where ATP production takes place. In essence, where reasoning, learning and memory abound, there is an abundance of DHA.

Research has shown that omega-3 fatty acids can play important role in the integration and regulation of both the structure and neurological function of the brain. DHA is proven essential to pre- and postnatal brain development, whereas EPA seems more influential on behavior and mood. Both DHA and EPA generate neuroprotective metabolites. Studies have shown that DHA and EPA supplementation ameliorates deficit/hyperactivity disorder (ADHD), autism, dyspraxia, dyslexia, and aggression. Studies have also shown that DHA and EPA supplementation improved mood, alertness, attention and overall cognitive performance and decreased mental fatigue.

DHA and EPA may be supplied in various forms, from various sources, such as, for example, fish oil, krill oil, and flaxseed oil. Existing commercial sources of DHA and EPA may be used in supplements and compositions of the invention, e.g., Schiff, Nordic Naturals, Nature Made, Puritan, etc.

Preferred amounts of DHA and EPA used within supplements and compositions of the invention include about 100 mg to about 2000 mg, about 250 mg to about 1500 mg, or about 250 mg to about 1000 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, about 1000 mg, about 1200 mg, about 1500 mg, about 1800 mg, or about 2000 mg, or any intervening amount therein.

6. Lipids and Phospholipids

Lipids and phospholipids are important in the formation and maintenance of neuronal synapses. In addition, choline-based supplements are important because they are metabolic precursors to acetylcholine and phosphatidyl choline, and may also increase monoaminergic neurotransmission, which is important for improving cognitive ability.

Illustrative examples of lipids or phospholipids suitable for use in the supplements and compositions contemplated herein include, but are not limited to, L-alpha glycerylphosphoryl choline, citicoline, or a choline salt, e.g., choline bitartrate. In one embodiment, a supplement comprises one or more of glycerylphosphoryl choline, citicoline, or a choline salt. In another embodiment, a supplement comprises glycerylphosphoryl choline, citicoline, and a choline salt.

a. L-Alpha Glycerylphosphorylcholine (Alpha GPC)

Alpha GPC is a natural choline compound found in the brain and in milk. It is also a parasympathomimetic acetylcholine precursor, and has been found to enhance memory and cognition. Alpha GPC rapidly delivers choline to the brain across the blood brain barrier and is a biosynthetic precursor of the acetylcholine neurotransmitter, which is one of the brain's most important neurotransmitters associated with heightened states of attention, improved memory and learning. Alpha GPC has also been shown to increase the levels of monoaminergic neurotransmitters dopamine and serotonin.

Alpha GPC suitable for use in supplements and compositions of the invention may be supplied in natural and synthetic forms, and from existing commercial sources, e.g. Vitaminshoppe, Ray Sahelian, GNC, IAS, etc.

Preferred amounts of alpha GPC used within supplements and compositions of the invention include about 100 mg to about 2000 mg, about 250 mg to about 1500 mg, or about 400 mg to about 1000 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, about 1000 mg, about 1200 mg, about 1500 mg, about 1800 mg, or about 2000 mg, or any intervening amount therein.

b. Choline Bitartrate

Choline is the natural precursor for the neurotransmitter acetylcholine. The bitartrate salt is a highly bioavailable form of choline and demonstrates efficient transport of choline across the blood brain barrier. Choline supplementation has demonstrated effects in humans including improvement in memory, thinking ability and serial-type learning in clinical studies.

Choline bitartrate and other bioavailable choline salts suitable for use in supplements and compositions of the invention may be supplied in natural and synthetic forms, and from existing commercial sources, e.g., Puritan, Vitacost, Vitaminshoppe, IAS, etc.

Preferred amounts of choline bitartrate and other bioavailable choline salts used within supplements and compositions of the invention include about 100 mg to about 2000 mg, about 250 mg to about 1500 mg, or about 400 mg to about 1000 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, about 1000 mg, about 1200 mg, about 1500 mg, about 1800 mg, or about 2000 mg, or any intervening amount therein.

c. Citicoline

Citicoline, also known as CDP-Choline, is a psychostimulant. It is an intermediate in the generation of phosphatidylcholine, which itself can be converted to acetylcholine. Citicoline supplementation has been shown to increase levels of dopamine, dopamine receptors, and acetylcholine. Studies have shown CDP-choline supplementation may help improve memory, mental focus and mental energy (reduced mental fatigue). Citicoline has neuroprotective effects that may be due to preservation of cardiolipin and sphingomyelin, preservation of arachidonic acid content of phosphatidylcholine and phosphatidylethanolamine, partial restoration of phosphatidylcholine levels, synaptic construction, and stimulation of glutathione synthesis and glutathione reductase activity. Citicoline's effects may also be explained by the reduction of phospholipase A2 activity.

Citicoline suitable for use in supplements and compositions of the invention may be supplied in natural and synthetic forms, and from existing commercial sources, e.g. Ray Sahelian, Vitaminshoppe, GNC, etc.

Preferred amounts of citicoline used within supplements and compositions of the invention include about 100 mg to about 2000 mg, about 250 mg to about 1500 mg, or about 400 mg to about 1000 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, about 1000 mg, about 1200 mg, about 1500 mg, about 1800 mg, or about 2000 mg, or any intervening amount therein.

7. Amino Acids

Amino acids are biologically important organic compounds made from amine (—NH₂) and carboxylic acid (—COOH) functional groups, along with a side-chain specific to each amino acid. The key elements of an amino acid are carbon, hydrogen, oxygen, and nitrogen, though other elements are found in the side-chains of certain amino acids. About 500 amino acids are known which can be classified in many ways.

Amino acids perform critical biological roles in the nervous system including, but not limited to neurotransmitter synthesis, synapse formation, and synaptic plasticity. Accordingly, various amino acids may have important roles in increasing cognitive abilities.

Illustrative examples of amino acids suitable for use in the supplements and compositions contemplated herein include, but are not limited to acetyl L-carnitine, L-carnitine, L-theanine, and L-metheanine. In one embodiment, a supplement comprises acetyl L-carnitine, L-carnitine, L-theanine, and L-metheanine. In a particular embodiment, a supplement comprises one or more of acetyl L-carnitine, L-carnitine, L-theanine, or L-metheanine.

a. Acetyl-L-Carnitine (ALCAR)

ALCAR is an acetylated form of L-carnitine that can efficiently cross the blood brain barrier. ALCAR may also be classified as a B vitamin. ALCAR may have higher bioavailability than L-carnitine because it may enter cells more efficiently than L-carnitine. L-carnitine usually requires an increase in carbohydrates and insulin to efficiently enter cells. ALCAR is known to produce energy from long chain fatty acids, and ALCAR enhances cognitive ability because it increases the production and release of acetylcholine in the brain. Studies have shown that ALCAR supplementation enhances mood, memory, visuo-spatial capacity, and vocabulary recall. Research has shown that ALCAR can also act as a neuroprotective agent because of its strong antioxidant properties and because it is linked to increases in the neuronal survival factor, nerve growth factor (NGF).

ALCAR suitable for use in supplements and compositions of the invention may be supplied in natural and synthetic forms, and from existing commercial sources, e.g. Puritan, iHerb, Vitaminworld, Dr. Weill, etc.

Preferred amounts of ALCAR used within supplements and compositions of the invention include about 100 mg to about 2000 mg, about 250 mg to about 1500 mg, or about 400 mg to about 1000 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, or about 1000 mg, or any intervening amount therein.

b. L-Carnitine

L-carnitine is a naturally occurring quaternary ammonium compound and is an important contributor to cellular energy metabolism. L-carnitine may also be classified as a B vitamin. L-carnitine has strong antioxidant properties and its highest concentrations are found in the most active metabolic tissue, such as the myocardium, skeletal muscle, and brain. L-carnitine is less active than ALCAR, but is imbued with similar properties for improving cognitive ability and neuronal survival.

L-carnitine suitable for use in supplements and compositions of the invention may be supplied in natural and synthetic forms, and from existing commercial sources, e.g. Puritan, GNC, Dr. Vitamin, etc.

Preferred amounts of L-carnitine used within supplements and compositions of the invention include about 100 mg to about 2000 mg, about 250 mg to about 1500 mg, or about 400 mg to about 1000 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, or about 1000 mg, or any intervening amount therein.

c. L-Theanine

L-theanine is an amino acid and a glutamic acid analog commonly found in tea (infusions of Camellia sinensis), primarily in green and black teas. L-theanine can readily cross the blood brain barrier. Studies have also shown that L-theanine supplementation increases mental alertness, attention, and memory and may provide neuroprotective effects. In addition, while structurally related to the excitatory neurotransmitter glutamate, theanine only has weak affinity for the glutamate receptor on postsynaptic cells. Theanine may also increase GABA and dopamine levels and have a low affinity for AMPA, kainate and NMDA receptors. In particular embodiments, L-metheanine may be substituted for L-theanine.

L-theanine suitable for use in supplements and compositions of the invention may be supplied in natural and synthetic forms, and from existing commercial sources, e.g. GNC, Dr. Vitamin, Vitamin World, Vitamin Shoppe, etc.

Preferred amounts of L-theanine used within supplements and compositions of the invention include about 50 mg to about 300 mg, about 100 mg to about 300 mg, or about 150 mg to about 300 mg, or any intervening range therein. In particular preferred embodiments, a supplement or composition comprises about 50 mg, about 100 mg, about 150 mg, about 200 mg, about 250 mg, or about 300 mg or any intervening amount therein.

8. Other Compounds

A number of additional compounds are useful in particular embodiments, such as, for example, nootropic agents. As used herein, the term “nootropic” refers to a smart drugs, memory enhancer, neuro enhancer, cognitive enhancer, and intelligence enhancer, such as a drug, supplement, nutriceutical, or functional foods that purportedly improves one or more cognitive abilities.

Illustrative examples of nootropics suitable for use in the supplements and compositions contemplated herein include, but are not limited to, racetams.

Illustrative examples of racetams suitable for use in the supplements and compositions contemplated herein include, but are not limited to, aniracetam, piracetam, and pramiracetam. In one embodiment, a supplement comprises aniracetam, piracetam, and pramiracetam. In a particular embodiment, a supplement comprises one or more of aniracetam, piracetam, or pramiracetam.

a. Racetams

Racetams are a class of nootropic compounds that are defined by their common pyrrolidone nucleus. Racetams are structurally similar but are a functionally diverse class of compounds, members of which positively modulate AMPA and glutamate receptors. Racetams also appear to increase cholinergic neurotransmission because some of them can increase the synthesis and/or release of acetylcholine. Racetam supplementation has also shown that these compounds improve mental functions such as cognition, memory, intelligence, motivation, attention, and concentration.

Racetams suitable for use in supplements and compositions of the invention may be supplied from existing commercial sources, e.g. IAS, Ray Sahelian, GNC, etc.

Preferred amounts of racetams used within supplements and compositions of the invention depend on which racetam is included (e.g., aniracetam, pramiracetam, piracetam, oxiracetam, etc.), and include about 100 mg to about 2000 mg, about 250 mg to about 1500 mg, or about 400 mg to about 1000 mg, or any intervening range therein. In particular preferred embodiments, a supplement or composition comprises about 100 mg, about 200 mg, about 300 mg, about 400 mg, about 500 mg, about 600 mg, about 700 mg, about 800 mg, about 900 mg, or about 1000 mg, or any intervening amount therein.

Table 1 illustrates certain preferred components of the supplements contemplated herein. It will be appreciated that supplements comprising all possible combinations of exemplary formulations in Table 1 are contemplated herein.

| | | | Exemplary | |
|---|---|---|---|---|
| Class | Type/Subtype | | formulation | Purported Role or Function |
| vitamins | B | B1 | thiamine | supports memory formation and consolidation |
| | | B5 | pantothenic acid | increase alertness |
| | | B9 | folate | reduce homocysteine |
| | | B12 | Methylcobalamin, | reduce homocysteine |
| | | | hydroxycobalamin, or | |
| | | | cyanocobalamin | |
| | D | D3 | cholecalciferol | binds Vitamin D receptor; multiple genes expressed, |
| | | | | including many involved with neurite extension; reduces |
| | | | | protein aggregation, inhibits Alzheimer's |

| | | | |
|---|---|---|---|
| alkaloids | xanthine | caffeine | anti-oxidant; improves alertness and memory formation |
| | alkaloid | | |
| | derivatives | | |
| | vinca alkaloid | Vinpocetine or | anti-inflammatory and nootropic |
| | derivatives | cyclopropylmethyl | |
| | | apovincaminate | |
| | Sesquiterpene/ | huperizine or | inhibits cholinesterase, therefore increases acetylcholine and |
| | other alkaloid | galantamine | memory formation, as well as focus |
| | derivatives | | |
| herbs | Rhodiola rosea | | MAO inhibitor; increases dopamine; also other effects |
| | Bacopa | | anti-oxidant; reduction of divalent metals |
| | monnieri | | |
| | Ginkgo biloba | | multiple mechanisms, including inhibition of thrombosis, |
| | | | inhibition of norepinephrine reuptake, and other less well |
| | | | characterized |
| | Panax ginseng | | multiple mechanisms |
| elements | magnesium | Mg threonate, | improves memory; affects NMDA receptor; antagonizes |
| | | Mg glycinate, | calcium |
| | | Mg oxide, | |
| | | Mg gluconate, or | |
| | | Mg citrate | |
| fatty | Omega-3 fatty | DHA or | synaptogenesis; reduces inflammation |
| acids | acids | EPA | |
| lipids & | choline | Alpha-GPC, | synaptogenesis; increases cholinergic transmission |
| phospholipids | | Choline bitartarate, | |
| | | or | |
| | | Citicholine | |
| amino | carnitine | ALCAR or | increase NGF levels |
| acids - | | L-carnitine | |
| derivs | theanine | L-theanine or | GABA support |
| | | L-metheanine | |
| other | racetams | aniracetam, | nootropics |
| | | piracetam, or | |
| | | pramiracetam | |

In various embodiments, supplements and compositions contemplated herein include, but are not limited to, one or more vitamins selected from the group consisting of Vitamin D3 and Vitamin B1, B5, B9, and B12; one or more alkaloids selected from the group consisting of caffeine, vinpocetine, cyclopropylmethyl apovincaminate, huperzine, and galantamine; and one or more herbs selected from the group consisting of Rhodiola rosea, Bacopa monnieri, Ginkgo biloba, and Panax ginseng.

Supplements may further comprise one or more minerals selected from the group consisting of Mg threonate, Mg glycinate, Mg gluconate, Mg citrate, and Mg oxide; one or more fatty acids selected from the group consisting of DHA and EPA; one or more lipids and phospholipids selected from the group consisting of L-alpha glycerylphosphorylcholine, citicoline, and a choline salt, e.g., choline bitartrate; one or more amino acids selected from the group consisting of acetyl L-carnitine, L-carnitine, L-theanine, and L-metheanine; and one or more racetams selected from the group consisting of aniracetam, piracetam, and pramiracetam.

In one embodiment, the supplement comprises vitamin D3, Vitamin B1, B5, B9, and B12, caffeine, vinpocetine, huperzine, Rhodiola rosea, Bacopa monnieri, Ginkgo biloba, and Panax ginseng. In one embodiment, the supplement comprises vitamin D3; Vitamin B1, B5, B9, and B12; caffeine; vinpocetine or cyclopropylmethyl apovincaminate; huperzine or galantamine; and Rhodiola rosea, Bacopa monnieri, Ginkgo biloba, and Panax ginseng.

In a particular embodiment, the supplement comprises vitamin D3, Vitamin B1, B5, B9, and B12, caffeine, vinpocetine, huperzine, Rhodiola rosea, Bacopa monnieri, Ginkgo biloba, and Panax ginseng, Mg threonate, DHA, L-alpha glycerylphosphorylcholine, acetyl L-carnitine, L-theanine, and aniracetam.

In a certain embodiment, the supplement comprises vitamin D3; Vitamin B1, B5, B9, and B12; caffeine; vinpocetine or cyclopropylmethyl apovincaminate; huperzine or galantamine; Rhodiola rosea, Bacopa monnieri, Ginkgo biloba, and Panax ginseng; Mg threonate, Mg glycinate, Mg gluconate, Mg citrate, or Mg oxide; DHA or EPA; L-alpha glycerylphosphorylcholine, citicoline, or a choline salt, e.g., choline bitartrate; acetyl L-carnitine or L-carnitine; L-theanine or L-metheanine; and aniracetam, piracetam, or pramiracetam.

The foregoing combinations are merely illustrative and not necessarily limiting. In various embodiments, other combinations of the supplement components shown in Table 1 are contemplated herein. For example, a supplement may comprise supplement components from at least 3, at least 4, at least 5 at least 6, at least 7 or at least 8 different classes shown Table 1.

Typically, supplements will comprise an amount of one more supplement components effective to improve one or more cognitive abilities and/or moods. In various embodiments, an effective amount is an amount sufficient to improve at least one cognitive ability, to improve cholinergic neurotransmission, to improve monoaminergic neurotransmission, and to improve synaptic formation, synaptic maintenance, and/or synaptic plasticity. Exemplary effective doses are provided in Table 2.

TABLE 1
Illustrative dosage ranges and dose levels for components of the
supplements contemplated herein. It will be appreciated that supplements comprising
all possible combinations of exemplary formulations and daily dose ranges and daily
doses in Table 2 are contemplated herein.

| | | | | Exemplary | Exemplary | | | | | | |
|---|---|---|---|---|---|---|---|---|---|---|---|
| | | | Exemplary | Daily Dose | Daily Dose | Exemplary | | Exemplary | | Exemplary | |
| Class | Type/Subtype | | formulation | Range | Range | Daily Dose | | Daily Dose | | Daily Dose | |
| vitamins | B | B1 | thiamine | 2.5 mg to | 10 mg to | 2.5 | mg | 10 | mg | 25 | mg |
| | | | | 25 mg | 25 mg | | | | | | |
| | | B5 | pantothenic acid | 100 mg to | 150 mg to | 200 | mg | 250 | mg | 250 | mg |
| | | | | 250 mg | 250 mg | | | | | | |
| | | B9 | methyl- | .4 mg to | .8 mg to | .8 | mg | 1 | mg | 1.5 | mg |
| | | | tetrahydrofolate, | 10 mg | 2.5 mg | | | | | | |
| | | | folate | | | | | | | | |
| | | B12 | Methylcobalamin, | .5 mg to | 5 mg to | 1 | mg | 1.5 | | 2.5 | |
| | | | hydroxycobalamin, or | 10 mg | 10 mg | | | | | | |
| | | | cyanocobalamin | | | | | | | | |
| | D | D3 | cholecalciferol | 500 IU to | 1000 IU | 1000 | IU | 1500 | IU | 5000 | IU |
| | | | | 5000 IU | to 5000 | | | | | | |
| | | | | | IU | | | | | | |

| | | | | | | | | | | |
|---|---|---|---|---|---|---|---|---|---|---|
| alkaloids | xanthine | caffeine | 25 mg to | 50 mg to | 50 | mg | 75 | mg | 100 | mg |
| | alkaloid | | 100 mg | 75 mg | | | | | | |
| | derivatives | | | | | | | | | |
| | vinca alkaloid | Vinpocetine or | 1 mg to | 2.5 mg to | 5 | mg | 7.5 | mg | 10 | mg |
| | derivatives | cyclopropylmethyl | 10 mg | 7.5 mg | | | | | | |
| | | apovincaminate | | | | | | | | |
| | Sesquiterpene/ | huperizine | 25 ug to | 50 ug to | 50 | ug | 75 | ug | 100 | ug |
| | other alkaloid | | 100 ug | 75 ug | | | | | | |
| | derivatives | galantamine | 2 mg to | 5 mg to | 2 | mg | 10 | mg | 24 | mg |
| | | | 24 mg | 15 mg | | | | | | |
| herbs | Rhodiola rosea | | 100 mg to | 250 mg to | 300 | mg | 400 | mg | 500 | mg |
| | | | 500 mg | 500 mg | | | | | | |
| | Bacopa | | 50 mg to | 200 mg to | 200 | mg | 300 | mg | 500 | mg |
| | monnieri | | 500 mg | 500 mg | | | | | | |
| | Ginkgo biloba | | 25 mg to | 60 mg to | 60 | mg | 120 | mg | 200 | mg |
| | | | 200 mg | 120 mg | | | | | | |
| | Panax ginseng | | 200 mg to | 300 mg to | 400 | mg | 500 | mg | 800 | mg |
| | | | 800 mg | 600 mg | | | | | | |
| elements | magnesium | Mg threonate, | 200 mg to | 250 mg to | 500 | mg | 600 | mg | 800 | mg |
| | | Mg glycinate, | 800 mg | 750 mg | | | | | | |
| | | Mg oxide, | | | | | | | | |
| | | Mg gluconate, or | | | | | | | | |
| | | Mg citrate | | | | | | | | |
| fatty | Omega-3 fatty | DHA or | 250 mg to | 400 mg to | 500 | mg | 1000 | mg | 1500 | mg |
| acids | acids | EPA | 1500 mg | 1000 mg | | | | | | |
| lipids & | choline | Alpha-GPC, | 250 mg to | 400 mg to | 500 | mg | 1000 | mg | 1500 | mg |
| phospholipids | | Choline bitartarate, | 1500 mg | 1000 mg | | | | | | |
| | | or | | | | | | | | |
| | | Citicholine | | | | | | | | |
| amino | carnitine | ALCAR or | 250 mg to | 400 mg to | 500 | mg | 1000 | mg | 1500 | mg |
| acids - | | L-carnitine | 1500 mg | 1000 mg | | | | | | |
| derivs | theanine | L-theanine or | 100 mg to | 150 mg to | 200 | mg | 250 | mg | 300 | mg |
| | | L-metheanine | 300 mg | 300 mg | | | | | | |
| other | racetams | aniracetam, | 250 mg to | 400 mg to | 500 | mg | 1000 | mg | 1500 | mg |
| | | piracetam, or | 1500 mg | 1000 | | | | | | |
| | | pramiracetam | | | | | | | | |

The foregoing exemplary formulations and dosages are merely illustrative and not necessarily limiting. In various embodiments, other combinations and dosages of supplement components can be formulated.

### D. Formulations and Compositions

The compositions contemplated herein can be used in the form of a supplement, for example, in solid, semi-solid, gel, or liquid form which contains the ingredients of the present invention in admixture with an organic or inorganic carrier or excipient suitable for external, enteral or parenteral applications. The supplement components may be individually or collectively formulated in a solid, semi-solid, gel, or liquid form along with one or more pharmaceutically acceptable carriers, diluents, or excipients. The supplement components may be supplied in many forms including, but not limited to pills, gummies, a bar, a shot, and a liquid, or any suitable combiantion thereof.

Compositions or supplements (i.e., medicaments) of the present invention include, but are not limited to pharmaceutical compositions. A “pharmaceutical composition” refers to a formulation of a supplement or composition contemplated herein with one or more pharmaceutically acceptable carriers, diluents or excipients generally accepted in the art for the delivery of the biologically active compounds to mammals, e.g., humans. There is virtually no limit to other reagents that may also be included in the compositions, provided that the additional reagents do not adversely affect the desired cognitive improvement.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In one embodiment, a supplement is formulated as a single discrete dosage form, i.e., one tablet, one volume of liquid, one mass of ointment, etc. For example, the supplement is formulated such that all the components are in a single formulation.

In another embodiment, a supplement is formulated in a plurality of dosage forms, i.e., two or more tablets, two or more volumes of liquid, two and/or more masses of ointment, etc. For example, the supplement is formulated such that part of the supplement components are in a solid tablet form and the remainder of the components are in a liquid form. In another non-limiting example, the supplement is formulated such that the supplement components are in two, three, four, or five or more tablets or other solid dosage forms. In another non-limiting example, the supplement is formulated such that the supplement components are in two, three, four, or five or more liquid dosage forms. In another non-limiting example, the supplement is formulated such that the supplement components are in any combination of two, three, four, or five or more solid, semi-solid, gel, or liquid dosage forms.

The supplements contemplated herein may be formulated for use in a single unit package. A “single unit package” is one that contains one discrete pharmaceutical dosage form. A “unit dose package” is one that contains the particular dose of the supplement for the patient. A single unit package is also a unit dose or single dose package if it contains the particular dose of the supplement ordered for the patient. A unit dose package could, for example, contain two tablets of a supplement, each tablet comprising all the supplement components, or each tablet comprising some of the supplement components, which together comprise the complete supplement.

The supplement components may be formulated as one composition, so as to facilitate and encourage patient compliance. For example, in one embodiment, a single liquid formulation may comprise one or more vitamins selected from the group consisting of Vitamin D3 and Vitamin B1, B5, B9, and B12; one or more alkaloids selected from the group consisting of caffeine, vinpocetine, cyclopropylmethyl apovincaminate, huperzine, and galantamine; and one or more herbs selected from the group consisting of Rhodiola rosea, Bacopa monnieri, Ginkgo biloba, and Panax ginseng, and one or more pharmaceutically acceptable carriers, diluents, or excipients.

In another embodiment, a single liquid formulation may comprise one or more vitamins selected from the group consisting of Vitamin D3 and Vitamin B1, B5, B9, and B12; one or more alkaloids selected from the group consisting of caffeine, vinpocetine, cyclopropylmethyl apovincaminate, huperzine, and galantamine; and one or more herbs selected from the group consisting of Rhodiola rosea, Bacopa monnieri, Ginkgo biloba, and Panax ginseng; one or more minerals selected from the group consisting of Mg threonate, Mg glycinate, Mg gluconate, Mg citrate, and Mg oxide; one or more fatty acids selected from the group consisting of DHA and EPA; one or more lipids and phospholipids selected from the group consisting of L-alpha glycerylphosphorylcholine, citicoline, and a choline salt, e.g., choline bitartrate; one or more amino acids selected from the group consisting of acetyl L-carnitine, L-carnitine, L-theanine, and L-metheanine; and one or more racetams selected from the group consisting of aniracetam, piracetam, and pramiracetam; and one or more pharmaceutically acceptable carriers, diluents, or excipients.

It will be recognized that delivery of a complete supplement can be accomplished by the use of combinations of commercially available dietary supplements. For example, a supplement comprising vitamin D3, Vitamin B1, B5, B9, and B12, caffeine, vinpocetine, huperzine, Rhodiola rosea, Bacopa monnieri, Ginkgo biloba, and Panax ginseng, Mg threonate, DHA, L-alpha glycerylphosphorylcholine, acetyl L-carnitine, L-theanine, and aniracetam can be achieved with a combination of commercially available supplements.

In particular embodiments, using combinations of commercial products to achieve the complete supplement contemplated herein typically introduces additional components that do not adversely affect the activity of the supplement that improves an individual's cognitive ability and/or mood. In certain embodiments, the introduction of such additional components may not be desired, e.g., where the combination pushes particular components above the recommended maximum daily dosage or adversely affects the supplement's desired activity.

As disclosed herein, the supplement may be formulated into one or more “unit dosage” forms. Techniques for formulation and administration of drugs may be found in Remington: The Science and Practice of Pharmacy. 22^{nd} Edition. Pharmaceutical Press. 2012, which is incorporated herein by reference in its entirety. The nature of the formulation will depend on the intended route(s) of administration. Suitable routes of administration may, for example, include oral, transdermal, rectal, transmucosal (e.g., transnasal), intestinal, parenteral delivery, including intramuscular, subcutaneous and intramedullary injections as well as intrathecal, intravenous, intranasal, or intraocular injections. Preferably, the supplements described herein are administered orally.

The supplements described herein or subsets of supplement components may be manufactured by processes well known in the art, e.g., by means of conventional mixing, dissolving, granulating, dragee-making, levigating, emulsifying, encapsulating, entrapping or lyophilizing processes.

Thus, supplements or combinations of supplement components may be formulated for oral administration by combining the active agent(s) with pharmaceutically acceptable carriers suitable for oral delivery well known in the art. Such carriers enable the active agent(s) described herein to be formulated as tablets, powders, pills, bars, shots, gummies, dragees, caplets, lozenges, gelcaps, capsules, liquids, gels, syrups, slurries, suspensions and the like, for oral ingestion by a patient/subject to be treated. For oral solid formulations such as, for example, powders, capsules and tablets, suitable pharmaceutically acceptable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the cross-linked polyvinylpyrrolidone, agar, or alginic acid or a salt thereof such as sodium alginate.

The solid dosage forms can be coated or otherwise prepared to provide the advantage of prolonged action. For example, the tablets or pills can comprise both an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Solid dosage forms may be sugar-coated or enteric-coated using standard techniques, described for example in U.S. Pat. Nos. 4,786,505 and 4,853,230.

In particular embodiments, supplements or combinations of supplement components may be formulated for oral use using a solid excipient, optionally grinding the resulting mixture, and processing the mixture of granules, after adding suitable auxiliaries if desired, to obtain tablets or dragee cores. Suitable excipients include, but are not limited to, particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). As indicated above, if desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Formulations for oral administration also include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. Formulations for oral administration should typically be in dosages suitable for the chosen route of administration.

Liquid dosage formulations for oral administration may include pharmaceutically acceptable solutions, beverage, suspensions, syrups and elixirs. The liquid forms contemplated herein and comprising the supplement or combinations of supplement components include aqueous solutions, suitably flavored syrups, aqueous or oil suspensions, and flavored emulsions with edible oils such as cottonseed oil, sesame oil, coconut oil, or peanut oil as well as elixirs and similar administration vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin.

In particular embodiments, the supplement or combination of supplement components are formulated as a beverage or beverage concentrate adapted for oral administration with water or other liquids, such as juices, iced tea, tea, and soda.

Liquid preparations for oral administration may also be prepared as a dry product for reconstitution with water or other suitable liquids before use. Such liquid preparations may be prepared by conventional means with additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

In one embodiment, the supplement or combination of supplement components are formulated such that it may be added to any hot or cold beverage, for example, iced tea, hot water or hot tea.

In certain embodiments, the supplement or combination of supplement components are may also be provided as food additives. Food additives include, for example, any liquid or solid material that is intended to be added to a food product. This material can, for example, include an agent having a distinct taste and/or flavor or a physiological effect (e.g., the multicomponent formulations described herein or subsets of the components comprising such formulations). In various embodiments, the supplement or combination of supplement components contemplated herein can be added to a variety of food products.

As used herein, the phrase “food product” describes a material comprising protein, carbohydrate and/or fat, that is used in the body of an organism to sustain growth, repair and vital processes and to furnish energy. Food products may also contain supplementary substances such as minerals, vitamins and condiments. The phrase “food product” as used herein further includes a beverage adapted for human or animal consumption.

A food product containing the supplement or combination of supplement components contemplated herein can also include additional additives such as, for example, certain antioxidants, sweeteners, flavorings, colors, preservatives, nutritive additives such as vitamins and minerals, amino acids (i.e. essential amino acids), emulsifiers, pH control agents such as acidulants, hydrocolloids, antifoams and release agents, flour improving or strengthening agents, raising or leavening agents, gases and chelating agents, the utility and effects of which are well-known in the art.

Supplements or combinations of supplement components may also be formulated for administration by inhalation, the active agent(s) are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount.

In various embodiments, the supplements or combinations of supplement components may be formulated in rectal compositions such as suppositories or retention enemas, e.g., containing conventional suppository bases such as cocoa butter or other glycerides. Methods of formulating active agents for rectal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45), amphiphilic materials such as Suppocire AP and polyglycolized glyceride, and the like). The base is selected and compounded for a desired melting/delivery profile.

In particular embodiments, supplements or combinations of supplement components may be formulated for systemic administration (e.g., as an injectable) in accordance with standard methods well known to those of skill in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can contain formulatory agents such as suspending, stabilizing and/or dispersing agents. In certain embodiments, the supplements or combinations of supplement components can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. Such penetrants are generally known in the art. Injectable formulations and inhalable formulations are generally provided as a sterile or substantially sterile formulation.

Supplements or combinations of supplement components may also be formulated as a depot preparation. Such long acting formulations can be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The foregoing formulations are intended to be illustrative and not limiting. Using the teachings provided herein, other methods of formulating and/or delivering the supplement or combination of supplement components contemplated herein will be available to one of skill in the art.

### E. Administration and Dosing Schedules

Supplements contemplated herein may be administered as one or more solids, semi-solids, gels, or liquids, or combination thereof. For example, a complete supplement may be formulated for oral administration as a single tablet or capsule or as a combination of one or more tablets, capsules, or liquids or other dosage forms. The specific amount/dosage regimen will vary depending on the weight, gender, age and health of the individual; the formulation, the biochemical nature, bioactivity, bioavailability and the side effects of the supplement components and the number and identity of the components in the complete supplement.

In various embodiments, the supplements are self-administered, i.e., taken by the patient without medical or parental supervision. In some embodiments, administration of the supplement may be under the direction of a physician or adult if the individual taking the supplement is a minor or requires supervision.

In one embodiment, the complete supplement is administered to or taken by an individual at least one, at least two, at least three, at least four, or at least five times per day. The supplement may be administered in a single dosage form or one or more dosage forms. In particular embodiments, the supplement is taken with meals. In one embodiment, the supplement is taken or administered at least one, at least two, at least three, at least four, or at least five times per day in a convenient beverage form.

In other embodiments, the complete supplement is formulated into a plurality of dosage forms, each of which may be taken at least one, at least two, at least three, at least four, or at least five times per day. Each supplement component may be taken the same number of times at the same time per day or each supplement component may independently be taken at least one, at least two, at least three, at least four, or at least five times per day and at different times than other supplement components. In either case, the individual will take at least one complete dose of the supplement each day.

The supplement may be taken by the individual for at least a week, at least two weeks, at least three weeks, at least a month, at least two months, at least three months, at least four months, at least five months, at least six months, at least a year, at least two years, or more, or for any extended duration in order to further improve, maintain, or retain improved cognition. In particular embodiments, the level of cognitive ability of the individual taking the supplement may play a role in determining the length of use.

### F. Methods of Use

The supplements contemplated herein can be used to improve the cognitive brain function and/or moods of humans. Supplements and compositions contemplated herein can be used to synergistically enhance an individual's overall cognitive ability by improving or enhancing short term working memory, long-term memory, mental attention, mental alertness, mental concentration or focus, learning, memory consolidation and processing speed, reaction time, mental clarity, mental energy, and general reasoning.

Existing supplements for improving cognitive ability mainly are directed to help the elderly, those with neurological trauma, mild cognitive impairment (MCI) and/or neurodegenerative disease regain some of the lost cognitive ability due to age or injury. The presently contemplated methods are directed, in part, to the use of the supplements contemplated herein to improve cognitive ability in cognitively normal, young, and otherwise healthy individuals. In related embodiments, individuals may be young and otherwise healthy but also possess reduced cognitive ability due to various non-degenerative neurological disorders such as attention deficit disorder (ADD) and attention deficit hyperactive disorder (ADHD).

In various embodiments, contemplated methods comprise administering supplements contemplated herein to improve cognitive ability and/or moods by increasing structural and/or functional characteristics of the central nervous system related to cognition, such as, for example, increasing cholinergic and/or monoaminergic neurotransmission, increasing synapse formation, increasing synaptic strength, increasing the maintenance of synapses, increasing synaptic plasticity, increasing neuronal cell survival, decreasing neuronal cell death, and/or providing neuroprotective effects.

Subjects/individuals that may benefit from the methods described herein include individuals that are cognitively normal, young, and otherwise healthy individuals or individuals that may be young and otherwise healthy but also possess reduced cognitive ability due to various non-degenerative neurological disorders such ADD and ADHD. In particular embodiments the individuals taking the supplements may be professionals such as business executives, scientists, people generally on demanding assignments and even students, or simply those that want to maintain a high level of cognitive function, or improve their existing cognitive abilities. The present invention contemplates that the supplement is suitable for use in subjects about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, or about 45 years of age or any age range therein.

In one embodiment, a method for improving one or more cognitive abilities comprising administering a supplement contemplated herein to a subject is provided. Without wishing to be bound to any particular theory, it is contemplated that the present inventors have discovered a surprising combination of supplement components that together improve the cognitive abilities of a cognitively normal, younger and healthier population. The supplements improve cognition by improving one or more of the following cognitive abilities: short term working memory, long-term memory, mental attention, mental alertness, mental concentration or focus, learning, memory consolidation and processing speed, reaction time, mental clarity, mental energy, and general reasoning. In addition, the supplement may improve moods that are counterproductive to improving cognition, such as depression, fatigue, confusion, lack of focus, and anxiety, which can further lead to an improvement in cognitive ability.

In one embodiment, a method of improving mental concentration or focus comprising administering a supplement contemplated herein to a subject is provided. In a particular embodiment, a method of improving learning and memory comprising administering a supplement contemplated herein to a subject is provided. In a certain embodiment, a method of improving mental attention or mental alertness and/or decreasing mental fatigue comprising administering a supplement contemplated herein to a subject is provided.

In a particular embodiment, a method of improving cholinergic neurotransmission comprising administering a supplement contemplated herein to a subject is provided. Acetylcholinergic synaptic transmission is recognized as being important in mental attention processes, in learning and memory, and in other cognitive processes. The supplements contemplated herein comprise various components that increase cholinergic and particularly, acetylcholinergic synaptic transmission, e.g., B vitamins, huperzine A (or galantamine), Ginkgo biloba, alpha GPC, citicoline, choline bitartrate, ALCAR, L-carnitine, and racetams. The supplements improve acetylcholinergic synaptic transmission by increasing levels of the transmitter acetylcholine, through increased release or acetylcholinesterase inhibition, by increasing acetylcholine receptor expression, etc. Increasing acetylcholinergic synaptic transmission may also increase the synaptic plasticity of acetylcholinergic synapses, thereby allowing for improved synaptic maintenance and stronger synaptic connections.

In a particular embodiment, a method of improving monoaminergic neurotransmission comprising administering a supplement contemplated herein to a subject is provided. Monoamine neurotransmitters are neurotransmitters and neuromodulators that contain one amino group that is connected to an aromatic ring by a two-carbon chain (—CH2-CH2-). Monoamine neurotransmitters and neuromodulators include histamine, dopamine, noradrenaline (norepinephrine), adrenaline (epinephrine), serotonin (5-HT), melatonin, β-phenylethylamine, tyramine, tryptamine, octopamine, 3-iodothyronamine, and thyronamines. Specific transporter proteins called monoamine transporters transport monoamines in or out of a cell. After release into the synaptic cleft, monoamine neurotransmitter action is ended by reuptake into the presynaptic terminal. There, they can be repackaged into synaptic vesicles or degraded by the enzyme monoamine oxidase (MAO), which is a target of monoamine oxidase inhibitors, a class of antidepressants. The supplements contemplated herein comprise various components that increase monoaminergic synaptic transmission, e.g., Rhodiola rosea, Mg, L-theanine, L-metheanine, and racetams. The supplements improve monoaminergic synaptic transmission by increasing levels of the monoaminergic neurotransmitters, such as, for example, glutamate, dopamine, and serotonin; through increased release of monoaminergic neurotransmitters; through MOA inhibition; by increasing monoaminergic receptor expression, etc. Increasing monoaminergic synaptic transmission may also increase the synaptic plasticity of monoaminergic synapses, thereby allowing for improved synaptic maintenance and stronger synaptic connections.

In one embodiment, a method of improving synapse formation or maintenance comprising administering a supplement contemplated herein to a subject is provided. The supplements contemplated herein increase synaptic activity through various pathways and mechanisms. Synaptic activity is known to promote synaptic formation and increase the strength of synaptic connections. Use and strengthening of the synaptic connections improves the maintenance of synaptic connections, which is important in various cognitive tasks, e.g., learning, memory, etc.

In a particular embodiment, supplements contemplated herein can be used to mitigate or ameliorate in a mammal one or more symptoms associated with non-degenerative neurological disorders such as attention deficit disorder (ADD) and attention deficit hyperactive disorder (ADHD). ADD and ADHD are generally characterized by lack of attention and focus, and the supplements contemplated herein, including but not limited to those that increase cholinergic neurotransmission, are contemplated to improve cognitive abilities in such subjects.

Illustrative examples of symptoms associated with ADD and ADHD include, but are not limited to, inattentiveness, lack of concentration or focus, over-activity, impulsivity, or a combination thereof.

In particular embodiments, the method contemplated herein comprise measuring the cognitive ability and/or moods of the individual taking the supplement. Cognitive ability may be assessed before supplementation and throughout the period of supplementation at either regular or irregular intervals. The initial cognitive assessment may serve as a baseline to measure the improvement in cognitive ability provided by supplementation contemplated herein. In addition, the individual receiving the supplement may be compared against a subject whose cognitive ability is similar to the initial cognitive ability of the individual receiving the supplement.

Methods for measuring cognitive ability may be given by a psychologist or qualified professional either in person or remotely. In addition, cognitive ability can be assessed using computerized assessment programs. Cognitive abilities may be measured using any art-accepted method, including for example, testing for working memory such as by using the digit span test, testing for executive function including multi-tasking with multi-sensory input, and testing for attention and focus; e.g., using word list tests, using an “app” such as Memtrax, using a computer-based test of memory such as available from Cogstate or others, or using standard neuropsychological tests such as the CVLT (California Verbal Learning Test) or MMSE (mini-mental state examination).

### G. Kits

In one embodiment, the complete supplement may be formulated in a single unit dosage form.

In particular embodiments, the supplement components may each be formulated individually, for example, in multiple dosage forms such that a subject is able to select the particular individual components and the quantities thereof to suit its particular needs. Even, when formulated individually, subject compliance can be improved and convenience afforded by providing the components in an integrated kit or packaging system. For example, where the supplement components are individually formulated a kit can comprise one or more packages containing some or all of the components.

Supplement components may be bundled together in various packaging systems e.g., a pack or dispenser device, such as an FDA approved kit, that can contain one or more unit dosage forms that collectively comprise the complete supplement.

The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as further detailed above.

The packaging system or kit can be constructed to facilitate administration on a particular treatment schedule wherein tablets or combinations of tablets are provided in blisterpack rows labeled with the time of administration.

It will be appreciated that these kits/packaging systems are intended to be illustrative and not limiting. Using the teachings provided herein, numerous alternative packaging/dispensing systems will be available to provide the supplements contemplated herein.

In addition, the packaging systems/kits optionally include labeling and/or instructional materials providing directions (i.e., protocols) for the practice of the methods or use of the supplements of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A supplement comprising:
a) one or more vitamins selected from the group consisting of vitamin B and vitamin D;
b) one or more alkaloids selected from the group consisting of caffeine, vinpocetine, and huperzine; and
c) one or more herbs selected from the group consisting of Rhodiola rosea, Bacopa monnieri, Panax ginseng, and Gingko biloba.

2. The supplement of claim 1, wherein the vitamin D is vitamin D3.

3. The supplement of any one of claims 1 to 2, wherein the supplement comprises one or more B vitamins selected from the group consisting of vitamin B1 (thiamine), vitamin B5 (panthothenic acid), vitamin B9 (folate), methylcobalamin, hydroxocobalamin, and cyanocobalamin.

4. The supplement of any one of claims 1 to 3, wherein the supplement comprises the B vitamins: thiamine, panthothenic acid, and methylcobalamin.

5. The supplement of any one of claims 1 to 3, wherein the supplement comprises the B vitamins: thiamine, panthothenic acid, and hydroxocobalamin.

6. The supplement of any one of claims 1 to 3, wherein the supplement comprises the B vitamins: thiamine, panthothenic acid, and cyanocobalamin.

7. The supplement of any one of claims 4 to 6, wherein the supplement comprises folate.

8. The supplement of any one of claims 1 to 7, wherein the supplement comprises caffeine, vinpocetine, and huperzine.

9. The supplement of any one of claims 1 to 7, wherein the supplement comprises caffeine, cyclopropylmethyl apovincaminate, and huperzine.

10. The supplement of any one of claims 1 to 7, wherein the supplement comprises caffeine, cyclopropylmethyl apovincaminate, and galantamine.

11. The supplement of any one of claims 1 to 7, wherein the supplement comprises caffeine, vinpocetine, and galantamine.

12. The supplement of any one of claims 1 to 11, wherein the supplement comprises Rhodiola rosea, Bacopa monnieri, Panax ginseng, and Gingko biloba.

13. The supplement of any one of claims 1 to 12, further comprising one or more Omega-3 fatty acids.

14. The supplement of claim 13, wherein the one or more Omega-3 fatty acids are selected from the group consisting of docosahexaenoic acid (DHA) and eicosapentaenoic acid (EPA).

15. The supplement of claim 14, wherein the supplement comprises DHA and EPA.

16. The supplement of claim 14, wherein the supplement comprises DHA.

17. The supplement of claim 14, wherein the supplement comprises EPA.

18. The supplement of any one of claims 1 to 15, further comprising a lipid or phospholipid.

19. The supplement of claim 18, wherein the lipid or phospholipid is L-alpha glycerylphosphorylcholine (Alpha-GPC), choline bitartarate, or citicholine.

20. The supplement of claim 19, wherein the lipid or phospholipid is Alpha-GPC.

21. The supplement of claim 19, wherein the lipid or phospholipid is choline bitartarate.

22. The supplement of claim 19, wherein the lipid or phospholipid is citicholine.

23. The supplement of any one of claims 1 to 22, further comprising one or more amino acids.

24. The supplement of claim 23, wherein the supplement comprises the one or more amino acids selected from the group consisting of L-theanine, L-metheanine, L-carnitine, and acetyl L-carnitine (ALCAR).

25. The supplement of claim 23, wherein the supplement comprises the one or more amino acids selected from the group consisting of L-theanine and L-metheanine

26. The supplement of claim 25, wherein the supplement comprises L-theanine.

27. The supplement of claim 23, wherein the supplement comprises the one or more amino acids selected from the group consisting of L-carnitine and ALCAR.

28. The supplement of claim 27, wherein the supplement comprises ALCAR.

29. The supplement of any one of claims 1 to 28, further comprising aniracetam, piracetam, and pramiracetam.

30. The supplement of any one of claims 1 to 28, further comprising aniracetam, piracetam, or pramiracetam.

31. The supplement of any one of claims 1 to 30, wherein the supplement comprises aniracetam.

32. The supplement of any one of claims 1 to 31, further comprising magnesium threonate, magnesium glycinate, magnesium oxide, magnesium gluconate, or magnesium citrate.

33. The supplement of any one of claims 1 to 32, wherein the supplement comprises magnesium threonate.

34. A supplement comprising thiamine, panthothenic acid, folate, methylcobalamin, ALCAR, vitamin D3, caffeine, vinpocetine, huperzine, Rhodiola rosea, Bacopa monnieri, Panax ginseng, Gingko biloba, DHA, Alpha-GPC, L-theanine, aniracetam, and magnesium threonate.

35. The supplement of any one of claims 1 to 34, wherein the supplement is formulated as a single unit dosage form or as a combination of supplement components in a plurality of unit dosage forms.

36. The supplement of claim 35, wherein the dosage form selected from the group consisting of: solid, semi-solid, powder, liquid, effervescent, rapidly dissolving in liquid, sublingual, time release, chewable, gummy, gum, lozenges, encapsulated, and tablet.

37. A method for improving cholinergic neurotransmission in a subject comprising administering the subject the supplement of any one of claims 1 to 36.

38. A method of for improving monoaminergic neurotransmission in a subject comprising administering the subject the supplement of any one of claims 1 to 36.

39. A method for improving synaptic formation or maintenance in a subject comprising administering the subject the supplement of any one of claims 1 to 36.

40. A method for increasing the concentration or mental focus in a subject comprising administering the subject the supplement of any one of claims 1 to 36.

41. The method of any one of claims 37 to 40, wherein the subject has at least one symptom associated with attention deficit disorder (ADD) and attention deficit hyperactive disorder (ADHD), sensory integration disorder, any learning or attention disorder (e.g., dyslexia), any cognitive disorder, or other disorders associated with learning, memory, or cognitive performance.

42. The method of any one of claims 37 to 41, wherein administration of the supplement to the subject results in a decrease in inattentiveness, over-activity, impulsivity, or a combination thereof.

43. The method of any one of claims 37 to 42, wherein the supplement is administered at least one, at least two, at least three, at least four, or at least five times a day.

44. The method of claim 43, wherein the supplement is self-administered.

45. The method of claim 43 or 44, wherein the supplement is orally administered.

46. The method of claim 43 or 44, wherein the supplement is formulated for transdermal administration.

47. The method of claim 35, wherein the one or more supplement components are administered the same time or different times.

48. The method of any one of claims 37 to 47, wherein the supplement is administered for at least one week, at least two weeks, at least one month, at least two months, at least three months, at least four months, at least five months, at least six months, at least one year or more.

49. A kit comprising the supplement of any one of claims 1 to 36.

50. The kit of claim 49, wherein the supplement is packaged as a single formulation.

51. The kit of claim 49, wherein the supplement is packaged as multiple-component formulations, wherein each supplement component is individually packaged.

52. The kit of claim 49, wherein the supplement is formulated in a solid dosage form.

53. The kit of claim 49, wherein the supplement is formulated in a liquid dosage form.

54. The kit of claim 49, wherein the supplement is a multiple-component formulation comprising both solid and liquid dosage forms.

55. A supplement according to any one of claims 1 to 36 that has one or more of the purported roles or functions disclosed in Table 1.
